(19) United States
(12) Patent Application Publication
(10) Pub. No.: US 2010/0173287 A1
(43) Pub. Date: Jul. 8, 2010

(54) METHOD OF ANALYZING NUCLEIC ACID

(75) Inventors: Yukie Nakashima, Shiki (JP); Keiichi Nagai, Higashiyamato (JP)

Correspondence Address: MATTINGLY & MALUR, P.C., 1800 DIAGONAL ROAD, SUITE 370, ALEXANDRIA, VA, 22314, US

(73) Assignee: HITACHI, LTD., Tokyo (JP)

(21) Appl. No.: 12/090,701

(22) PCT Filed: Mar. 7, 2006
(86) PCT No.: PCT/JP2006/304357

§ 371 Date: Apr. 18, 2008

(30) Foreign Application Priority Data
Oct. 20, 2005 (JP) 2005-306162

(51) Int. Cl. C12Q 1/68

(52) U.S. Cl. 435/6

(57) ABSTRACT

According to the present invention, stable amplification of a small amount of nucleic acid and analysis of the same with good sensitivity can be realized by improving efficiency of hybridization primers or probes with a probe. Specifically, the present invention relates to a method of analyzing nucleic acid comprising: a step of hybridizing at least one type of a first probe comprising a 1^{st} sequence complementary to one strand of double-strand nucleic acid, a 2^{nd} sequence complementary to the other strand thereof with the double-strand nucleic acid, and a 3^{rd} sequence that binds the 1^{st} sequence and the 2^{nd} sequence; and a step of hybridizing at least one type of a second probe with the double-strand nucleic acid.

### TECHNICAL FIELD

The present invention relates to a method of analyzing nucleic acid, wherein a small amount of nucleic acid is analyzed with good sensitivity. More specifically, the present invention relates to a method of analyzing nucleic acid, wherein a small amount of nucleic acid is analyzed with good sensitivity by carrying out partial disruption of a higher-order structure of double-strand nucleic acid that serves as a template so as to improve hybridization efficiencies of primers and probes.

### BACKGROUND ART

A major task in the post-sequence era involves functional genome studies for the pursuit of gene functions. For such studies, techniques involving DNA chips (herein collectively referred to as “DNA chips” for purposes of explanation, such DNA chips including “DNA arrays” on which DNAs are arrayed and immobilized on a basal plate and DNA chips in different forms such as fiber bundle forms and bobbin forms), SNP analysis, and rapid analysis of protein interaction are used. In addition, genomic drug discovery has been underway based on the information obtained by the above techniques. Under such circumstances, gene expression analysis and mutation analysis account for a significant part of genome studies.

Such “DNA chips” refer to microarrays that were developed approximately 10 years ago, in which a 20- to 100-mer nucleotide, cDNA, BAC clone DNA, or the like is arranged on a basal plate at a high density. With the use of DNA chips, it is possible to examine a variety of qualitative and quantitative changes in a nucleic acid sample by carrying out hybridization between a DNA chip and a DNA or RNA specimen for sequencing, analysis of gene mutation or SNP, measurement of gene expression levels or copy numbers, DNA methylation analysis, and the like (see Non-Patent Document 1). In addition, DNA chips are involved in an innovative technology that allows observation of expression of a number of genes at many temporal points. Thus, it is possible to readily confirm the presence or absence of a gene to be examined or functions of such gene. In addition, it is possible to carry out exhaustive measurement of thousands to tens of thousands of genes. Thus, DNA chips represent an important means for carrying out studies. It is believed that great progress will be made in genome functional analysis following genomic structure analysis with the use of DNA chips, resulting in changes in studies of medicine, drug discovery, and organ regeneration. As a result of the Human Genome Project, new genes with known base sequences but with unknown functions were successively discovered. It is expected that such functional analysis of new genes will progress with the addition of nucleotide sequences of such genes as reference sequences. In the cases of studies of disease-related genes, methods comprising examining genes one by one have so far been available. Thus, it has been very difficult to analyze a group of genes related to multifactor diseases because the number of such diseases is much greater than that of single gene diseases. However, it is believed that the use of DNA chips will result in a great progress in studies involving exhaustive analysis of causative genes of diseases that have been studied with difficulty (such diseases including diabetes, Alzheimer disease, cardiovascular diseases, and rheumatism) for elucidation of the mechanisms in the development of such diseases. Further, it is expected that, based on findings obtained by the above studies, drug discovery can be achieved with very good efficiency compared with conventional cases, resulting in the realization of the development of promising drugs in the short term.

It is said that single nucleotide polymorphism (SNP) analysis will lead to the elucidation of risks of diseases and the genetic backgrounds of such diseases (see Non-Patent Document 2). It is also said that it will become possible to elucidate genes related to drug resistance by scanning genes of pathogens (see Non-Patent Document 3) and to detect the drug resistance of a detected pathogen prior to the initiation of treatment in the future. Also in the development of anticancer drugs, it is expected that the development speed be accelerated by examining influences on genes when allowing a drug to react in vitro and comparing the results with those obtained by using conventional drugs.

People engaging in basic and applied life science in various fields of bioengineering, agricultural science, and medical science regard PCR methods as effective experimental techniques that allow the supply of protein or gene information in a rapid and accurate manner. It is possible to carry out studies that differ in terms of experimental materials (such as microorganisms, animals, and plants) with the use of the same PCR method if study purposes are the same. The main characteristics of PCR include amplification of a specific region of complex DNA such as genomic DNA. PCR is a reaction in which DNA synthesis is caused by DNA polymerase with the use of two types of primers. Thus, a DNA region to be synthesized is a region to be identified with a primer, and such region alone is amplified. Each primer has the base sequence of a specific region of either one strand of template double-strand DNA. The 3′ end of one of the primers faces the other primer. DNA polymerase requires primers for the initiation of the synthesis reaction and causes elongation reaction by adding dNMP to the 3′ end of each primer in the presence of dNTPs under constant-pH conditions, resulting in a logarithmic increase of an amplification region.

In the cases of many gene analysis techniques (e.g., a sequencing, SNP analysis, mutation analysis, and expression analysis) that have been developed along with the progress in the Human Genome Project, it is often necessary to first carry out gene amplification in order to secure detection sensitivity. With the use of PCR methods, existing difficult problems have been easily solved in many cases. Thus, PCR methods play important roles in studies of basic and applied life science.

Non-Patent Document 1: BIO technology journal, vol. 5, No. 4, 394-396 (2005)

### Non-Patent Document 2: Wang D G, et al., Science, 280, 1077-82 (1998)

### Non-Patent Document 3: Winzeler E A, et al., Science, 281, 1194-97 (1998)

### DISCLOSURE OF THE INVENTION

### Problem to be Solved by the Invention

In general, it is necessary to prepare excessive amounts of probes relative to the amount of a sample when carrying out gene analysis comprising a step of hybridizing a probe with a sample in a specific manner. When a hybridization reaction takes place on a solid phase, it is difficult to detect signals in some cases because reaction efficiency usually less than reactions using a liquid phase. In addition, upon gene mutation analysis, it is essential to first amplify a gene fragment by PCR or the like. However, in general, it is difficult to stably amplify a gene fragment from a small amount of a nucleic acid sample.

It is an objective of the present invention to realize stable amplification and analysis with high sensitivity of a small amount of nucleic acid by improving the efficiency of hybridization between primers or probes and a template.

### Means for Solving Problem

A single-strand oligo (DNA, RNA, PNA, or chimeric oligo) that is complementary to a sequence other than a probe sequence that recognizes a detection target region of a nucleic acid sample is synthesized and then added in an excessive amount to a nucleic acid sample. The resultant is heated at 94° C., followed by cooling to room temperature. Single strands of the nucleic acid sample obtained by thermal denaturation tend to be rewound into the original double-strand form during cooling to room temperature, however, during which the complementary single-strand oligo that has been previously added in an excessive amount is first hybridized with one strand of such nucleic acid. Thus, the single strands cannot be rewound to form a complete double strand. The thus treated nucleic acid sample is more unstable than complete double-strand nucleic acid. Such unstable nucleic acid sample is added to a basal plate on which a probe sequence that recognizes a detection target region has been immobilized or to a solution containing a probe sequence that recognizes a detection target region, followed by hybridization. Accordingly, the nucleic acid sample that has been previously destabilized is more smoothly hybridized with a probe than complete double-strand nucleic acid that has not been destabilized.

The present invention has been established based on the above findings. According to the present invention, an oligo sequence that binds to a region other than a region to which a probe binds is first hybridized with a sample such that the double-strand structure or intramolecular structure of a nucleic acid sample is disrupted. Thus, effects that allow “a probe to be hybridized with a nucleic acid sample with ease” are provided.

### EFFECTS OF THE INVENTION

According to the present invention, upon gene mutation analysis or expression analysis with the use of hybridization reaction, the efficiency of hybridization a double-strand nucleic acid sample with a detection probe is improved so that analysis can be carried out with high sensitivity. Alternatively, upon nucleic acid fragment amplification involving elongation reaction caused by polymerase, the efficiency of hybridization of a small amount of a sample template with a primer is promoted, and thus stable gene amplification can be carried out.

### BRIEF DESCRIPTION OF THE DRAWINGS

FIG. 1 shows a positional relationship among a template DNA sequence, a first probe, and a second probe for explanation of the present invention.

FIG. 2 shows hybridization promotion effects (shown as fluorescence detection results) according to the present invention.

FIG. 3 shows structures of a first probe used in the present invention.

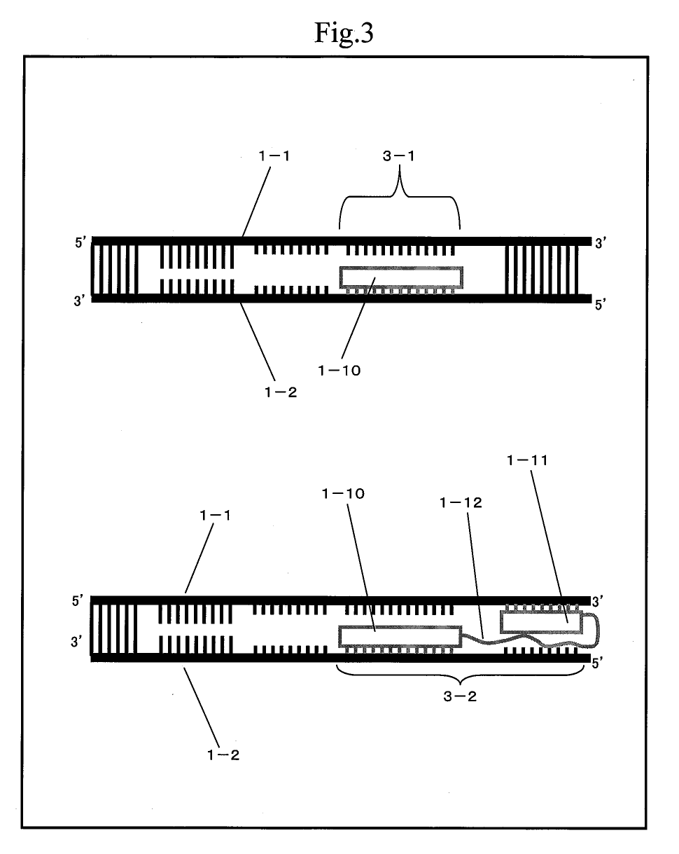

FIG. 4 shows effects of destabilization of a template nucleic acid according to the present invention.

FIG. 5 shows structures of a first probe used in the present invention.

FIG. 6 shows a structure of a first probe used in the present invention.

FIG. 7 shows a conceptual diagram of a conventional DNA chip using a single-strand nucleic acid sample.

FIG. 8 shows an example in which effects of the present invention can be obtained upon DNA chip analysis.

FIG. 9 shows a conceptual diagram of a conventional DNA chip using a double-strand nucleic acid sample.

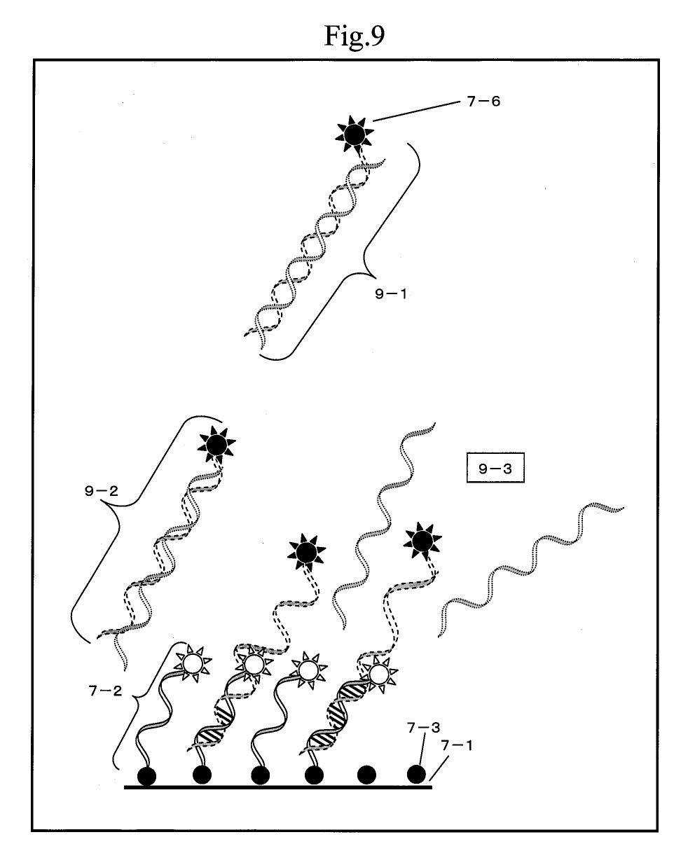

FIG. 10 shows a conceptual diagram of a DNA chip to which the double-strand nucleic acid sample has been applied according to the present invention.

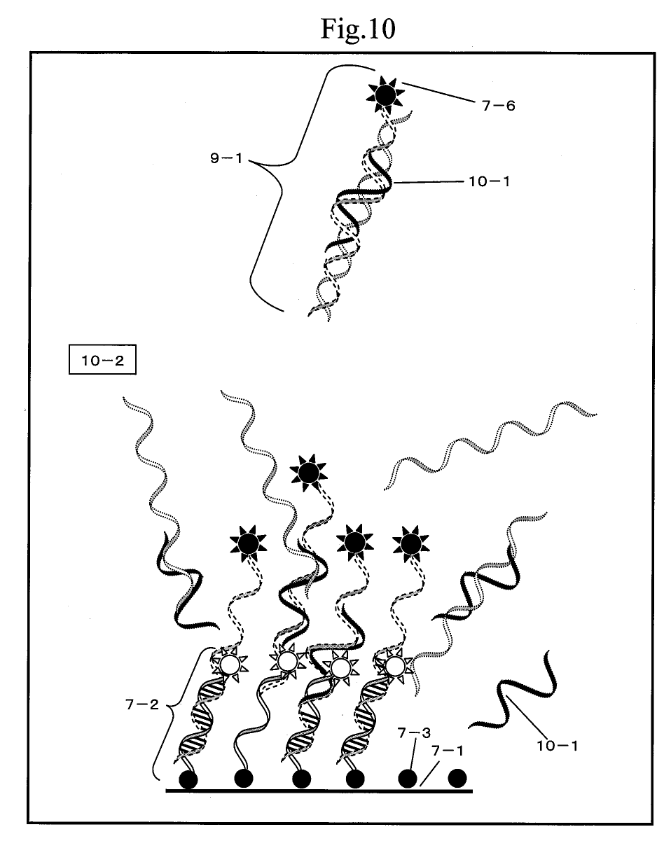

FIG. 11 shows a schematic view of bead chips.

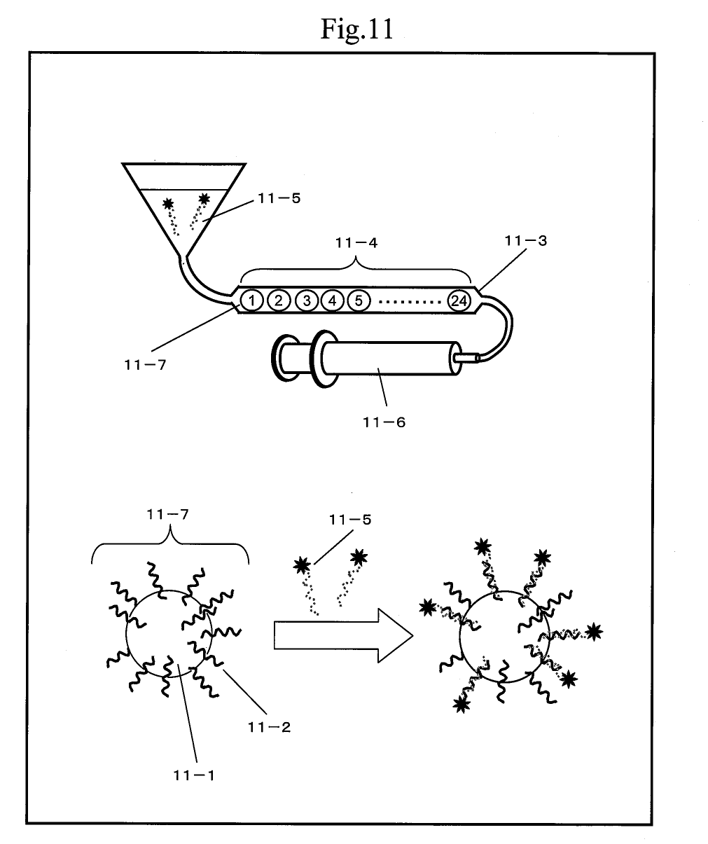

FIG. 12 shows a conceptual diagram of immobilization of a first probe to a glass bead.

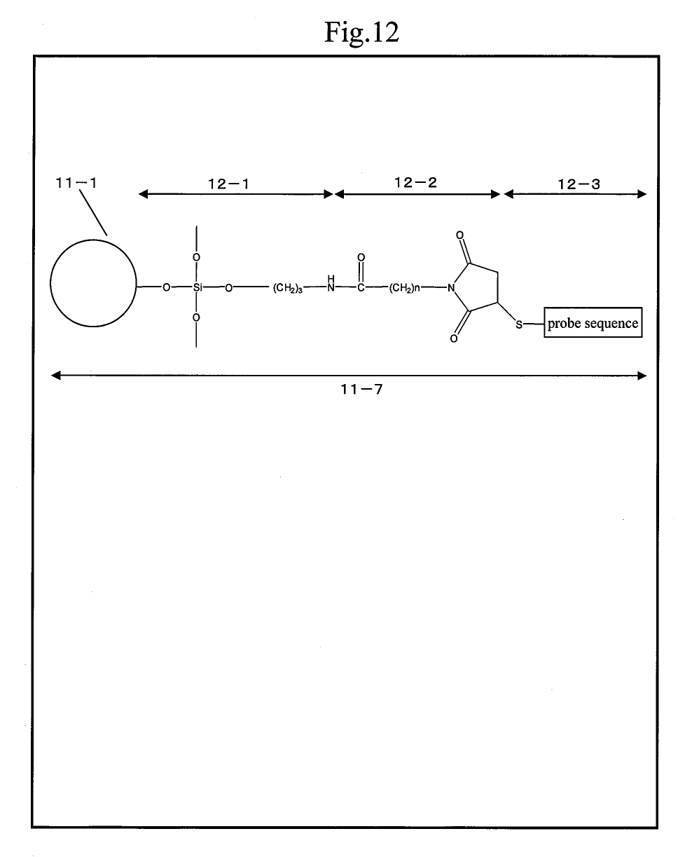

FIG. 13 shows a positional relationship among a template DNA sequence, a first probe, and a second probe for explanation of the present invention.

FIG. 14 shows hybridization promotion effects (based on comparison of PCR product amounts) of the present invention.

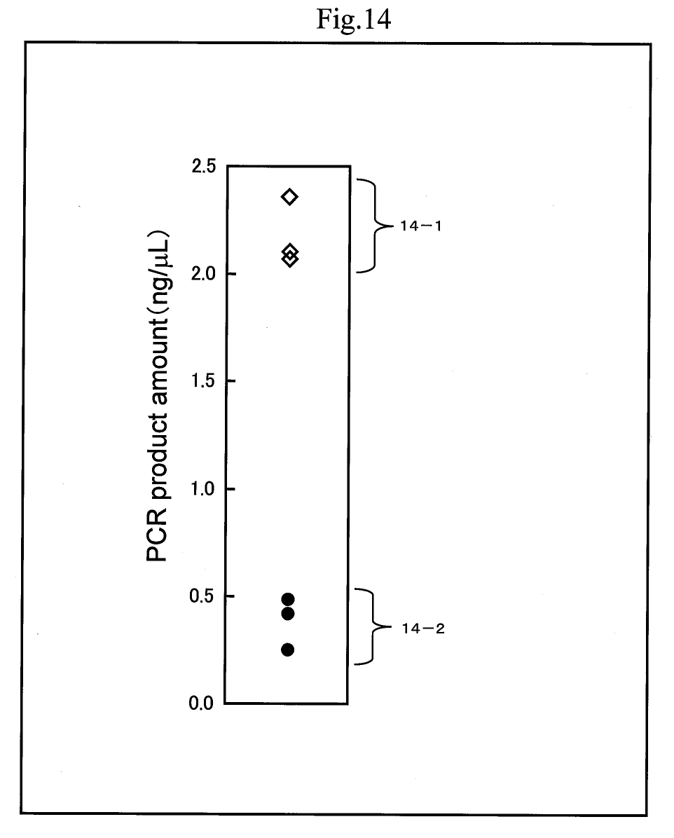

FIG. 15 shows PCR efficiency promotion effects (calculated based on PCR product amounts) of the present invention.

FIG. 16 shows PCR efficiency promotion effects (real-time PCR) of the present invention.

FIG. 17 shows PCR efficiency promotion effects (based on comparison of PCR product amounts) of the present invention.

FIG. 18 shows a positional relationship among a template DNA sequence, a first probe, and a second probe for explanation of the present invention.

FIG. 19 shows hybridization promotion effects (luminescence detection results) of the present invention.

### DESCRIPTION OF REFERENCE NUMERALS

1-1, 1-2 . . . Template double-strand DNAs
1-3, 1-4 . . . Primers
1-5 . . . A PCR product (amplification region)
1-6 . . . FITC label
1-7 . . . A second probe
1-8 . . . TEXAS RED label
1-9 . . . Labeled biotin
1-10 . . . A 1^{st} sequence
1-11 . . . A 2^{nd} sequence
1-12 . . . A 3^{rd} sequence
2-1 . . . Fluorescence intensity (background)
2-2 . . . Fluorescence intensity obtained with no addition
2-3 . . . Fluorescence intensity obtained with the addition of a first sequence
2-4 . . . Fluorescence intensity obtained with the addition of a first probe
3-1 . . . A 1^{st} sequence
3-2 . . . A first probe
4-1 . . . A stable double-strand
4-2 . . . A state of partially inhibited reassociation
4-3, 4-4 . . . Strands constituting a double-strand PCR product
4-5 . . . A first probe
5-1, 5-2 . . . Strands constituting template double-strand nucleic acid
5-3 . . . A 1^{st} sequence
5-4 . . . A 2^{nd} sequence
5-5 . . . A 3^{rd} sequence
5-6 . . . A 1′^{st} sequence
5-7 . . . A 2′^{nd} sequence
5-8 . . . A 3′^{rd} sequence
5-9 . . . A first probe designed to have a structure in which the center regions of a 3^{rd} sequence and a 3′^{rd} sequence are hybridized with each other
5-10 . . . A 3^{rd} sequence having a loop structure
5-11 . . . A first probe comprising a 3^{rd} sequence having a loop structure
6-1 . . . A first probe
6-2 . . . A 1^{st} sequence
6-3 . . . A 2^{nd} sequence
6-4 . . . A 3^{rd} sequence
7-1 . . . Basal plate
7-2 . . . A second probe (single-strand)
7-3 . . . Linker
7-4 . . . Label
7-5 . . . A single-strand nucleic acid sample
7-6 . . . Label
7-7 . . . A state of a hybridization reaction between a single-strand nucleic acid sample and a second probe
7-8 . . . A Stanford-type DNA chip
8-1 . . . DNA chip detection results (obtained with the use of single-strand nucleic acid as a sample)
8-2 . . . Background
8-3 . . . A signal resulting from hybridization
8-4 . . . A signal resulting from no hybridization
8-5 . . . DNA chip detection results (obtained with the use of denatured double-strand nucleic acid as a sample)
8-6 . . . DNA chip detection results (obtained with the use of a double-strand nucleic acid sample according to the present invention)
9-1 . . . A double-strand nucleic acid sample
9-2 . . . Reassociation
9-3 . . . A state of hybridization reaction between a double-strand nucleic acid sample and a second probe
10-1 . . . A first probe
10-2 . . . A state of hybridization reaction between a double-strand nucleic acid sample and a second probe (according to the present invention)
11-1 . . . Glass beads
11-2 . . . A second probe
11-3 . . . A capillary
11-4 . . . Glass beads arranged in a series in a capillary
11-5 . . . Fluorescence-labeled target nucleic acid
11-6 . . . A syringe
11-7 . . . a probe-immobilizing bead
12-1 . . . APS coat
12-2 . . . Crosslinker KMUS
12-3 . . . A probe sequence
13-1 . . . A 1^{st} sequence
13-2 . . . A 2^{nd} sequence
13-3 . . . A 3^{rd} sequence
13-4 . . . The 3′ end of a 2^{nd} sequence
13-5 . . . The 5′ end of a 1^{st} sequence
14-1 . . . PCR product amount (with the addition of a first probe)
14-2 . . . PCR product amount (control: without the addition of a first probe)
15-1 . . . PCR efficiency (first probe concentration: 3.4 pmol)
15-2 . . . PCR efficiency (first probe concentration: 6.8 pmol)
15-3 . . . PCR efficiency (first probe concentration: 10.2 pmol)
15-4 . . . PCR efficiency (control: without the addition of a first probe)
16-1 . . . Real-time PCR results (control: without the addition of a first probe)
16-2 . . . Real-time PCR results (with the addition of a first probe)
17-1 . . . PCR product concentration (general PCR reaction without the addition of a first probe)
17-2 . . . PCR product concentration (with the addition of a first probe)
18-1 . . . The second base of 3 bases constituting the 175^{th} codon of the TP53 gene
18-2 . . . A second probe sequence
18-3 . . . The 3′ end of a second probe sequence
18-4 . . . A mismatch base inserted into a second probe sequence
19-1 . . . Results of a BAMPER method (with the addition of a second probe alone)
19-2 . . . Results of a BAMPER method (with the addition of a first probe and a second probe)

This description includes part or all of the contents as disclosed in the description of Japanese Patent Application No. 2005-306162, which is a priority document of the present application.

### BEST MODE FOR CARRYING OUT THE INVENTION

According to the present invention, a method of analyzing nucleic acid, wherein a small amount of nucleic acid is analyzed with good sensitivity by carrying out partial disruption of a higher-order structure of double-strand nucleic acid that serves as a template so as to improve hybridization efficiencies of primers and probes, is provided.

Specifically, the present invention comprises: a step of hybridizing a first probe comprising a 1^{st} sequence complementary to one strand of double-strand nucleic acid, a 2^{nd} sequence complementary to the other strand thereof, and a 3^{rd} sequence that binds the 1^{st} sequence and the 2^{nd} sequence with the double-strand nucleic acid; and a step of hybridizing at least one type of a second probe with the double-strand nucleic acid.

According to the present invention, the 3^{rd} sequence constituting the first probe is preferably 10 mer to 100 mer and is not complementary to either sequence of the double-strand nucleic acid.

In addition, the binding region of the second probe on the double-strand nucleic acid is located between the binding region of the above 1^{st} sequence and the binding region of the above 2^{nd} sequence on the double-strand nucleic acid or in a region within 500 bases away from each end of such region. Particularly preferably, the 1^{st} sequence and the 2^{nd} sequence are each hybridized with a region that is at least 10 bases away from the end of the binding region of the second probe on the double-strand nucleic acid.

The above 3^{rd} sequence that constitutes the first probe may form a three-dimensional structure in a loop form as a result of intrastrand hybridization.

Further, at least two types of first probes are used. In such case, two types of first probes that have been separately hybridized with neighboring regions on the double-strand nucleic acid may form a complementary strand bond between their 3^{rd} sequences such that a three-dimensional ladder structure is formed.

According to the method of the present invention, the double-strand nucleic acid can be quantified by measuring the amount of the second probe hybridized with the double-strand nucleic acid.

For instance, the double-strand nucleic acid can be quantified based on the amount of fluorescence obtained by labeling the above second probe with a phosphor. Alternatively, the second probe is labeled with an enzyme selected from the group consisting of alkaline phosphatase, peroxidase, β-galactosidase, and luciferase. Then, the amount of the double-strand nucleic acid can be quantified based on the amount of luminescence or color development resulting from a reaction between the above enzyme and a substrate thereof. Further, the double-strand nucleic acid can be quantified based on the radiation quantity obtained by labeling the second probe with a radioactive isotope.

In one embodiment, a step of carrying out a complementary strand elongation reaction using the second probe hybridized with the double-strand nucleic acid may be further carried out.

According to the above method, it is preferable that the first probe have a structure in which at least one base of three bases at the 3′ end thereof is mismatched with a binding region of the first probe on the double-strand nucleic acid such that the first probe does not function as a member of a primer pair with respect to the second probe. It is also preferable that the first probe have a structure in which complementary strand elongation does not take place at the 3′ end thereof, or that a sequence that is not complementary to the binding region of the 1^{st} probe on the double-strand nucleic acid be added to the 3′ end thereof.

Alternatively, in order to inhibit complementary strand elongation starting from the 3′ end of the first probe, a hydroxyl group of the 3′ end may be modified or substituted with another functional group.

According to the method of the present invention, a second probe may be immobilized on a solid phase (e.g., the basal plate of a DNA chip or beads). With the use of DNA chips and bead arrays to which the techniques of the present invention are applied, gene analysis sensitivity can be significantly improved.

The method of the present invention can be used for mutation and polymorphism detection. Specifically, the method of the present invention comprises: a step of simultaneously adding the first probe and the second probe to a nucleic acid sample expected to have a mutation, such second probe being hybridized at the 3′ end thereof with a candidate region for the mutation; and a step of carrying out an elongation reaction with the use of the hybridized second probe. Thus, it is possible to judge whether or not the nucleic acid sample has a mutation site based on the results of the above elongation reaction.

For instance, a single base elongation reaction is carried out with the use of a second probe by applying a BAMPER method or the like. At such time, the type of base to be introduced is identified. Thus, it is possible to judge whether or not a neighboring base of the 3′ end of the second probe has a mutation.

In such case, the elongation reaction may be induced when at least two bases of the nucleic acid sample are complementary to at least two bases that exist at the 3′ end of the second probe. In addition, intramolecular hybridization in the second probe may be prevented by introducing a mismatch base into the second probe.

In another embodiment, the second probe comprises a pair of an upstream primer and a downstream primer used for amplification. The method of the present invention comprises a step of amplifying at least a partial region of the double-strand nucleic acid with the use of such primers. In such case, the 1^{st} sequence and the 2^{nd} sequence must be separately hybridized with regions neighboring the above primers on the double-strand nucleic acid. Preferably, the sequences are each hybridized with a region within 500 bases away from the end of the above region on the double-strand nucleic acid.

### EXAMPLES

The present invention is hereafter described in greater detail with reference to the following examples, although the technical scope of the present invention is not limited thereto.

First, general reaction operations used in the Examples of the present invention are explained below.

A hybridization experiment using a single-strand oligo serving as a probe and a template DNA sample is described. It is necessary to amplify a target region of a template nucleic acid sample by PCR prior to analysis. In this Example, one of the amplification primers was labeled with a phosphor (FITC used herein). The reaction container used was a polystyrene 96-well plate coated with streptavidin (PIERCE). The bottom and side surfaces of each well were coated with streptavidin, on which a detection probe (one side of which has been modified with biotin) can be immobilized as a result of binding. Specifically, a detection probe (modified with 5′-biotin/3′-TEXAS RED) (20 pmol) that had been dissolved in PBS (100 μL) was added to each well, followed by incubation at room temperature for 1 hour. Then, washing with PBS was carried out.

The above FITC-modified sample DNA (PCR product) was subjected to thermal denaturation at 95° C. for 5 minutes, followed by ice cooling. The resultant was adequately diluted with an N2S hybridization buffer (PIERCE) and then added to each well in which the aforementioned probe had been immobilized, followed by incubation at 60° C. for 1 hour. Thereafter, washing with an N2S hybridization buffer and PBS was carried out. Hybridization between the probe and the sample DNA was examined by detecting the fluorescence intensity. For fluorescence detection, an ARVO SX microplate reader (PerkinElmer) was used. In this Example, gene amplification primers and detection probes were modified with phosphors such as FITC and TEXAS RED. However, any phosphor can be selected, depending on the plate reader and fluorescence filter to be used. In addition, detection can be carried out by count measurement with the use of a radioisotope label, luminescence measurement with the use of an HRP or AP label, or the like, which can be selected in accordance with conditions.

### Example 1

### Application of the Present Invention to PCR

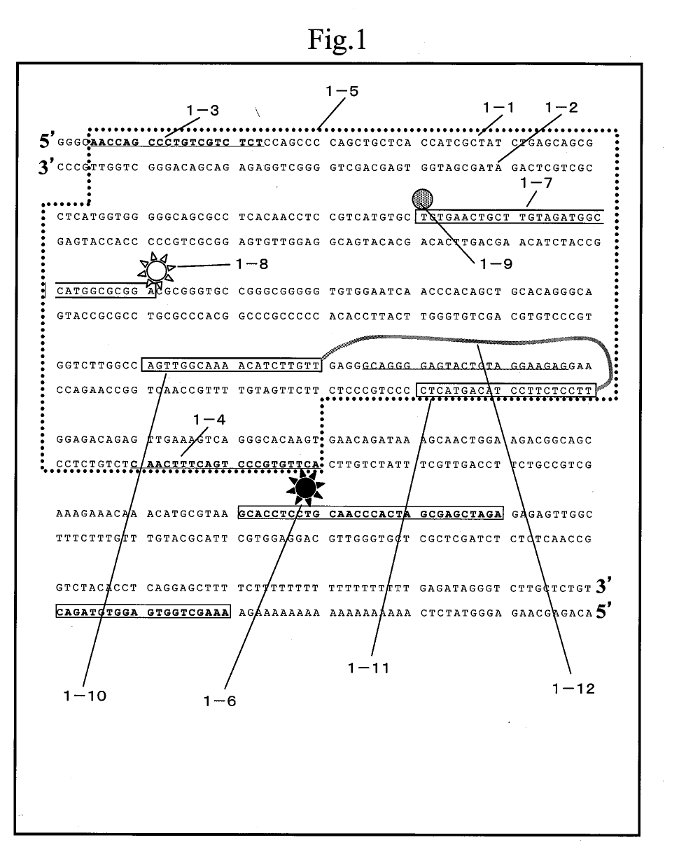

Herein, an example using the exon 5 of the TP53 gene is explained below. The sequence information can be obtained from the NCBI database (accession no. NT_{—}010718) (a part of the sequence (SEQ ID NO: 1) is shown in FIG. 1). The numerical reference 1-5 represents an amplification region obtained after PCR by allowing primers designed as designated by 1-3 and 1-4 to act on template double-strand DNA templates 1-1 and 1-2. ABI9700 thermal cycler (Applied Biosystems) used for elongation reaction. A PCR product was confirmed with the use of an SV1210 microchip electrophoresis system (Hitachi Electronic Engineering). All oligonualeotides were obtained from SIGMA Genosys. DNA polymerases used were obtained from QIAGEN. In addition, the reagent used was a well-known commercially available product. Template nucleic acid (human genomic DNA) was purchased from BIOCHAIN and then used.

General PCR procedures are described below. A genome sample (1 μL) prepared at 1×10⁻²⁰ mol/μL was added to a well of a 96-well plate. The plate was placed on ice. A Taq. DNA polymerase (0.2 μL) at 2.5 units/μL was mixed with a 2.5 mM dNTPs (4 μL) and a primer set (0.8 μL each: 25 pmol/μL) and adjusted to 100 μL per well with the addition of sterilized water. It is possible to change the above contents based on the same ratio. For instance, PCR may be carried out at a scale of 50 μL. The plate was sealed with an adhesive sheet and set in a thermal cycler. In order to denature double-strand genomic DNA, heating at 94° C. for 2 minutes was carried out and then a thermal cycle of 94° C. for 30 seconds, 50° C. for 30 seconds, and 72° C. for 1 minute was repeated 35 times. A PCR reaction solution was analyzed with a microchip electrophoresis system. Accordingly, actual PCR product was confirmed.

The reverse primer 1-4 used herein was fluorescence-labeled at the 5′ end thereof with an FITC 1-6. Thus, a PCR product 1-5 was also labeled. A second probe 1-7 was fluorescence-labeled at the 3′ end thereof with a TEXAS RED 1-8 and further labeled at the 5′ end thereof with a biotin 1-9, and then it was immobilized on an avidin-coated plate. When the PCR product 1-5 is trapped with the second probe 1-7, 1-5 is first subjected to thermal denaturation. In addition, the first probe of the present invention is simultaneously added. Such first probe comprises a region that is hybridized with a nucleic acid strand 1-2 (a 1^{st} sequence 1-10), a region that is hybridized with a nucleic acid strand 1-1 (a 2^{nd} sequence 1-11), and a 3^{rd} sequence 1-12 that is designed to bind the 3′ end of 1-10 and the 5′ end of 1-11. The sequence 1-12 is as follows: GATCTGCGATCTAAGTAAGCTTGGC (SEQ ID NO: 2).

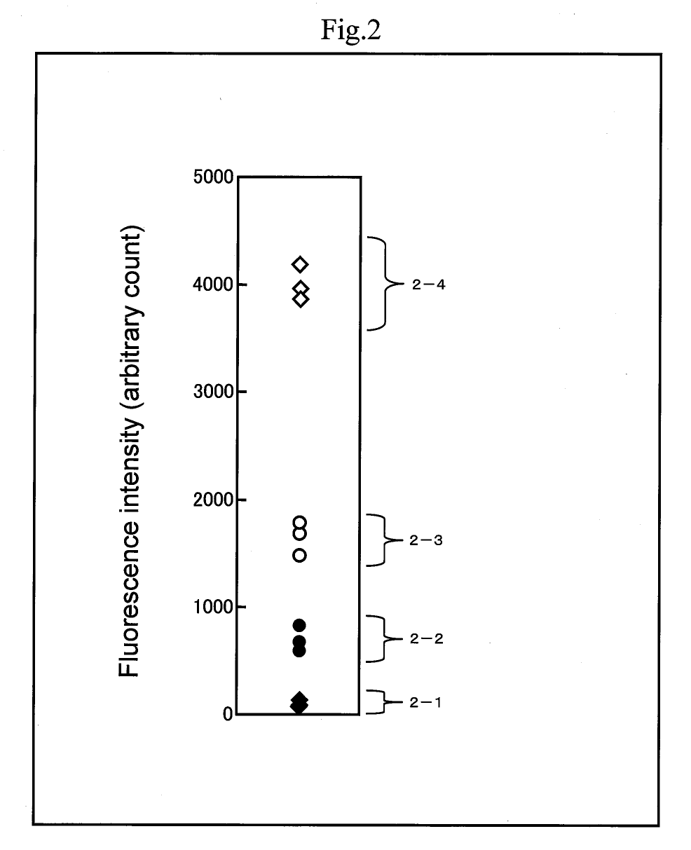

FIG. 2 shows the results of detection of actual FITC fluorescence intensity. In a case in which the aforementioned first probe was not added, a slight increase in the count designated by 2-2 was merely observed relative to the background 2-1. In fact, the PCR product 1-5 was not hybridized with the second probe 1-7 with high efficiency. On the other hand, in a case in which an oligo consisting of the 1^{st} sequence of the first probe was added, the count designated by 2-3 was obtained at a higher level than that of 2-2.

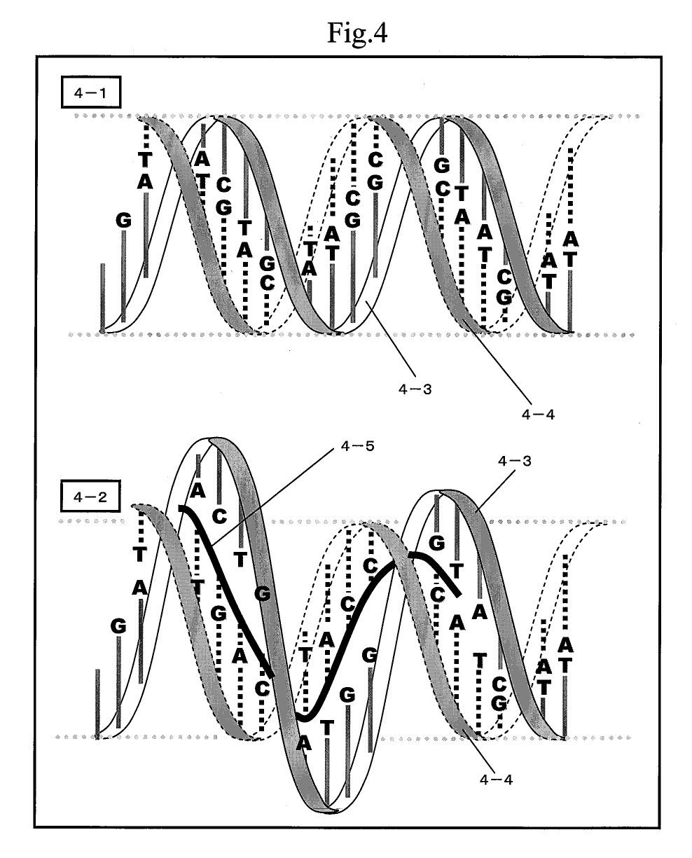

The above results are explained with reference to FIG. 4, which is a general conceptual diagram. In general, it is difficult for a double-strand PCR product (comprising a strand 4-3 and a strand 4-4), which has been previously thermally denatured, to remain denatured. Such PCR product tends to regain its original stable double-strand form (4-1). Meanwhile, it was considered that the above results were obtained as a result of the following events. A first probe 4-5 was partially hybridized with either one of strands of a double-strand PCR product (strand 4-4 in the figure). Then, as shown in 4-2, reassociation of a strand 4-3 and a strand 4-4 was partially inhibited. Accordingly, a second probe became likely to be hybridized with the PCR product. In a case in which a first probe 3-2 comprising a 1^{st} sequence 1-10, a 2^{nd} sequence 1-11, and a 3^{rd} sequence 1-12 was allowed to act on a PCR product 1-5 in the same manner described above, the count as designated by 2-4 was obtained, such count being much higher than that designated by 2-3 obtained by allowing a first sequence alone to act. That is, it was considered that both ends of a first probe (1-10 and 1-11) acted on a strand 1-1 and a strand 1-2, respectively. As a result, compared with the case in which a first probe 3-1 was allowed to act, it became more unstable double-strand structure would be formed.

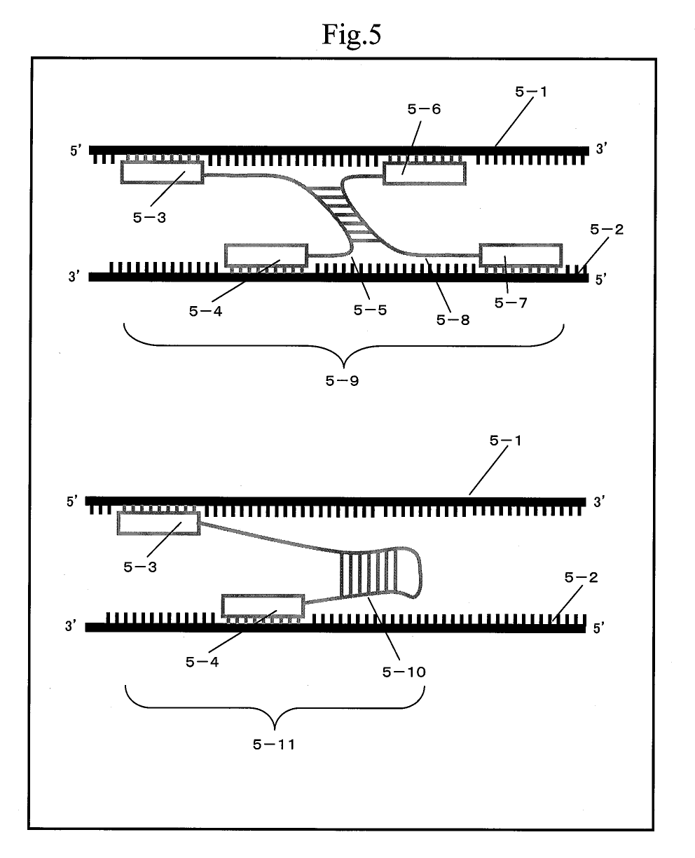

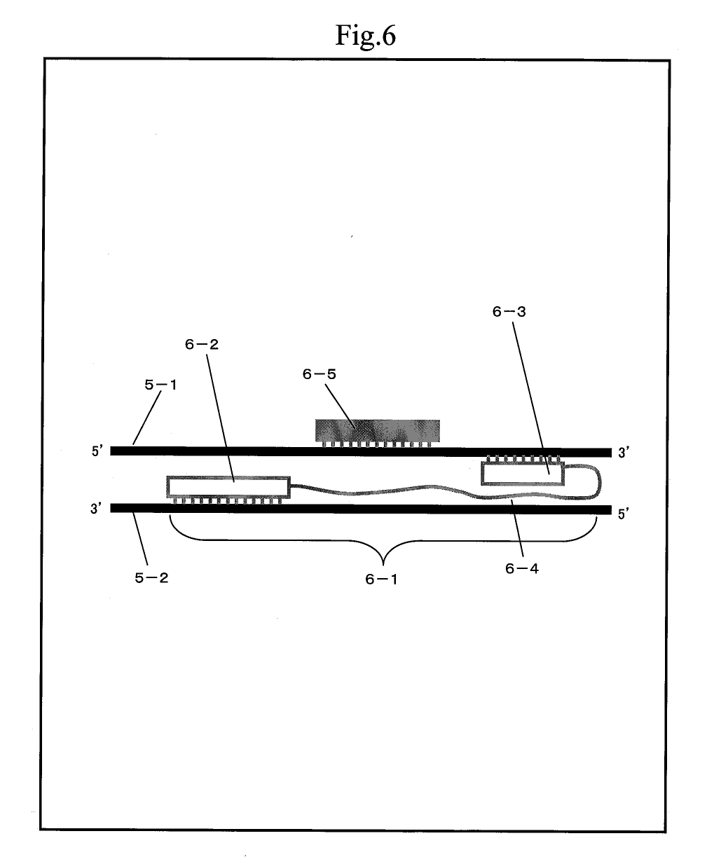

FIG. 5 shows an example of the structure of a first probe that exhibits a similar action. Specifically, in the case of FIG. 5, a first probe comprising a 1^{st} sequence 5-3, a 2^{nd} sequence 5-4, and a 3^{rd} sequence 5-5, and a first probe′ comprising a 1′^{st} sequence 5-6, a 2′^{nd} sequence 5-7, and a 3′^{rd} sequence 5-8 are allowed to simultaneously act on a template double-strand nucleic acid comprising 5-1 and 5-2 in the manner described above. Possible examples thereof include: a first probe 5-9 that is designed to have a structure in which the center region of the above 3^{rd} sequence and that of the above 3′^{rd} sequence are hybridized with each other; and a first probe 5-11 comprising a 1^{st} sequence 5-3, a 2nd sequence 5-4, and a 3^{rd} sequence 5-10, which is designed to have a loop structure of the 3^{rd} sequence 5-10 as a result of intrastrand hybridization. These first probes (3-2, 5-9, and 5-11) may be simultaneously hybridized with at least two regions of a nucleic acid sample. Alternatively, it is also possible to obtain a structure shown in FIG. 6 in which a 1^{st} sequence 6-2 and a 2^{nd} sequence 6-3 of a first probe 6-1 are placed such that they sandwich a second probe 6-5 from both sides of the second probe and the sequences are connected with a 3^{rd} sequence 6-4.

A 1^{st} sequence and a 2^{nd} sequence that constitute a first probe are hybridized with a region that is at least 10 bases, and preferably 50 bases, away from a second probe such that they do not prevent a second probe hybridization to a template. 3^{rd} sequences 1-12, 5-5, and 6-4 and a 3′^{rd} sequence 5-8 has not complementary sequence to prevent hybridization with template nucleic acid. Examples of such 3^{rd} sequence are not limited to the above sequences, and a 3^{rd} sequence depends on template sequences. The length of such sequence is 10 mer to 100 mer and preferably 50 mer or less.

### Example 2

### Application of the Present Invention to DNA Chips

Commercially available DNA chips are known two types: “Affymetrix-type” chips launched by Affymetrix; and “Stanford-type” chips devised by Patrick Brown et al. at Stanford University. The “Stanford-type” chips are simple chips obtained by sticking cDNA, synthesis oligo DNA, or the like, which have been previously prepared, on object glasses with the use of a thin pin. In a Stanford-type chip, a single spot contains large amounts of cDNA (double-strand DNA) and single-strand oligo DNA. cDNA or oligo DNA act as a reference probe for gene detection. Examples of similar types of DNA chips include “AceGene (registered trademark)” (DNA Chip Research Inc.), “CodeLink (registered trademark)” (GE Healthcare), and “IntelliGene (registered trademark)” (Takara Bio Inc.). The above “Affymetrix-type” chips are obtained by synthesizing a probe (single-strand oligo DNA) in a vertical direction on a basal plate. Examples thereof include “GeneChip (registered trademark)” (Affymetrix) and a microarray (Agilent Technologies Inc.). In addition to those obtained by the technology for immobilization of a probe on a basal plate, “Genopal (registered trademark)” (Mitsubishi Rayon Co., Ltd.) and an ECA chip (Electrochemical Array, TUM gene) can also be used. In any case, analysis results are influenced by the efficiency of hybridization between single-strand or double-strand DNA serving as a probe and a sample DNA. Also, the present invention can be applied to such cases.

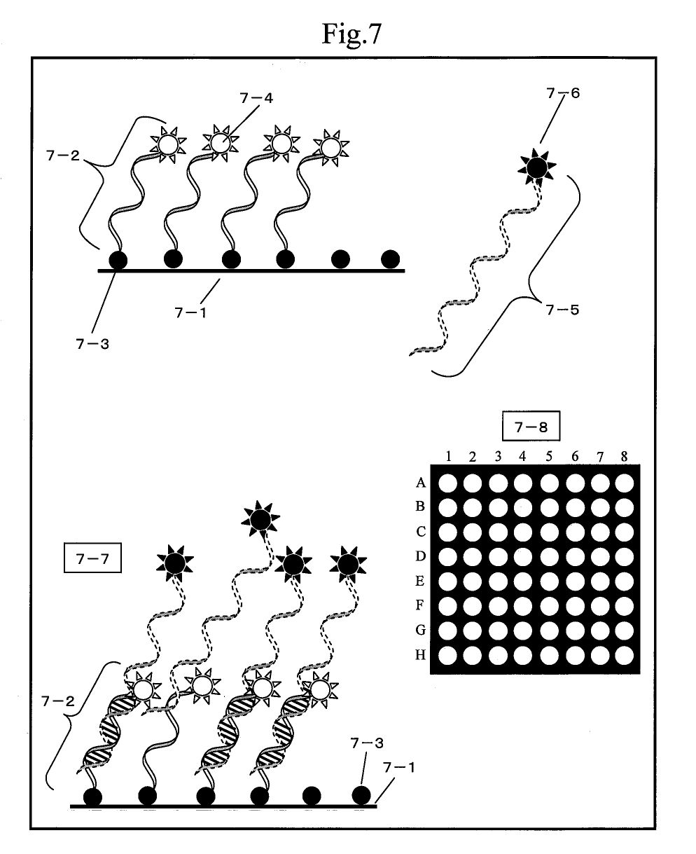

FIG. 7 shows results of verification of the efficiency of hybridization with the use of a general Stanford-type chip. There are 64 spots in total on a chip, such spot being formed as designated by 7-8. A probe specific to target gene is immobilized on each spot. Specifically, a second probe (single-strand) 7-2 is immobilized on a basal plate 7-1 via a linker 7-3. 7-2 may contain a phosphor or another label designated by 7-4 at the end thereof.

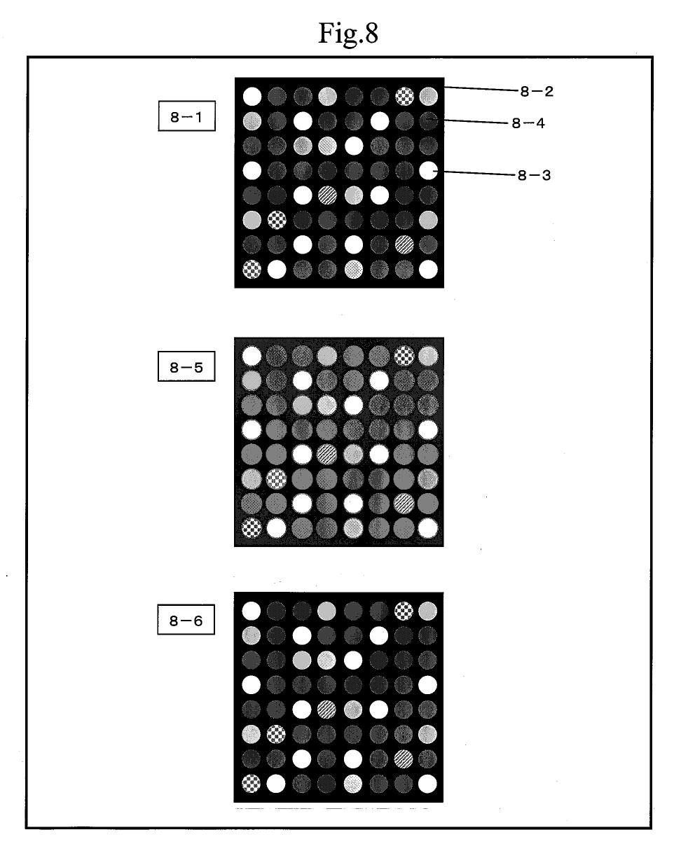

A single-strand nucleic acid sample (containing a phosphor or another label designated by 7-6 on one end thereof) designated by 7-5 was allowed to act on the above chip and a hybridization reaction with a second probe 7-2 was carried out in accordance with the above protocol. The results shown in FIG. 8-1 were obtained. When a sample 7-5 and a second probe 7-2 were hybridized with each other, a signal designated by 8-3 was obtained relative to a background 8-2. When a sample 7-5 and a second probe 7-2 were not hybridized with each other, a signal designated by 8-4 was obtained. It was considered that a hybridization reaction as shown in 7-7 took place in the above case with the use of 7-5 and 7-2.

Similar experiments were carried out with the use of, as a sample, a double-strand nucleic acid sample 9-1 (containing a phosphor or another label designated by 7-6 at one end thereof). In such case, 9-1 is first denatured by heating or alkaline treatment. However, in addition to the case of 9-2, a stable double-strand nucleic acid sample had high reassociation ability. So, the clear contrast between background and signal obtained in the case of 8-1 was not obtained in this case. The results were similar to those for the case of 8-5. This is because, as shown in 9-3, the efficiency of hybridization between a second probe 7-2 and a template 9-1 was not as high as that obtained in the case of 7-7. When an amount of a double-strand nucleic acid sample 9-1 added was increased in order to improve hybridization efficiency, the background derived from a label 7-6 that was located at the end of the nucleic acid expanded, and thus, a significant increase in the S/N ratio was not observed.

Likewise, the case to which the present invention was applied is descried below. In this case, double-strand nucleic acid was used as a sample (corresponding to 8-6). The double-strand nucleic acid sample 9-1 was previously hybridized with a first probe 10⁻¹ provided that, as described above, 10-1 would have the structure of 3-2, 5-9, 5-11, or 6-1. In accordance with the above protocol, a hybridization reaction was carried out using a second probe 7-2. The results shown in 8-6 were obtained. At such time, the sensitivity was comparable to that in the case of 8-1. That is, although the nucleic acid used as a sample had a double-strand structure as in the case of 9-1, it was considered that hybridization took place with good efficiency as shown in 10-2. It was demonstrated that the efficiency was comparable to that obtained in the case in which a nucleic acid sample was a single-strand (7-7). In general, when carrying out a hybridization reaction between a probe and a nucleic acid sample on a solid phase, it is necessary to first carry out alkaline denaturation of a nucleic acid sample so as to obtain a single-strand form. Further, it is also necessary to carry out column purification. However, these techniques are complicated. Alternatively, it is also possible to split the double-strand structure of a nucleic acid sample by thermal denaturation. Note that, in such case, a strong force works to rewind the strands so as to form a stable structure as described above, and thus it is difficult to cause the reaction of a nucleic acid sample as is and a probe with good efficiency. However, it was demonstrated that effects comparable to those obtained with a single-strand nucleic acid sample can be obtained with the use of the present invention, resulting in the same convenience as in the case of thermal denaturation of a double-strand nucleic acid sample. In this Example, the end of a second probe was labeled with a phosphor and then used. Also, a radioactive isotope may be used for labeling. In addition, an enzyme that develops color when reacting with a specific substance (such as alkaline phosphatase, peroxidase, or β-galactosidase) or the like may be used for labeling. For instance, in the case of labeling with alkaline phosphatase, a reaction with a substrate, which is nitroblue tetrazolium (NBT), was induced in a 5-bromo-4-chloro-3-indolyl phosphate (BCIP) solution for several hours such that purple color development was observed. Then, measurement and comparison in terms of color intensity was carried out such that the effects of the present invention were confirmed. Further, with the use of a chemiluminescent substrate, luminescence as a result of an enzyme reaction can be used for measurement.

### Example 3

### Application of the Present Invention to Bead Chips

DNA chips in plate form are generally used for various applications. However, particularly for medical and diagnostic applications, DNA chips are required to have improved sensitivity and to be available for rapid measurement. Medical applications does not need exhaustive analysis. So, it is enough to be used for up to 100 types of contents for medical analysis. However, it is desirable that it be possible to readily change the combination of contents to be tested. In addition, to prevent contamination among samples, DNA chips are required to be disposable. In order to comply with the above requirements, “bead chips” have been developed. They have a structure in which the above second probe 11-2 is immobilized on each glass bead 11-1 approximately 100 microns in diameter, and such glass beads are arranged in series in a capillary 11-3 or a groove of a microchip, which has almost same diameter. Such device is prepared in the following manner: each type of second probe is subjected to an immobilization reaction on the glass bead surface; and immobilized beads are selected one by one so as to be arranged as desired. Therefore, it is possible to identify the type of prove based on the order of beads. A sample solution containing fluorescence-labeled target nucleic acid 11-5 is fed in a reciprocating manner into a device with a syringe 11-6. Accordingly, a target nucleic acid 11-5 is trapped by a probe 11-2 on a bead 11-1. When a general DNA chip in a plate form is used, it takes time for target DNA to be dispersed so as to reach probe DNA. Thus, such reaction is very time-consuming. However, in the case of the above bead chip, the flow of a sample solution is disturbed, resulting in rapid completion of effective dispersion. Accordingly, rapid reaction and detection can be realized (Yoshinobu Kohara, “DNA Chip Jikken Maruwakari (Guide for DNA chip experimentation),” Yodosha Co., Ltd., 124-127 (2004)).

In practice, a glass bead 11-1 covered with an APS (aminopropyltrimethoxysilane) coat 12-1 and a thiol-end-modified oligo DNA 12-3 were immobilized to each other via a covalent bond formed with the use of a KMUS (N-(11-maleimidoundecanoyloxy)succinimide) 12-2, which is a crosslinker having NHS ester and maleimide on both ends thereof, respectively. Thus, a probe-immobilizing bead 11-7 was prepared. Herein, 24 types of second probes each corresponding to a base on the TP53 gene were used. Then, beads on which such probes had been immobilized were arranged one by one inside of a capillary 11-3. Subsequently, a hybridization reaction was carried out while 10 μL of a sample solution (1×10⁻¹⁰ M, 45° C., 4×SSC-0.1% SDS solution (FITC-labeled)) was fed into the capillary for 10 minutes, followed by washing in the following order: 0.2×SSC-0.03% SDS→0.05×SSC→water. Then, fluorescence measurement was carried out.

When the above 7-5 and 9-1 were used as samples and an experiment for hybridization between the samples and a bead corresponding to the exon 5 was conducted, the results similar to those of [Example 2] were obtained. Specifically, when comparison of hybridization efficiency (herein detected based on the fluorescence intensity) was carried out with the use of, as samples, a single-strand nucleic acid 7-5 and a double-strand nucleic acid 9-1, better results were obtained in the case of 7-5. Further, the fluorescence intensity in the case of the use of a template obtained by first hybridizing a double-strand nucleic acid 9-1 with a first probe 10-1 was the substantially same as that obtained in the case of the sample 7-5.

### Example 4

### Introduction of a Mismatch Sequence into a First Probe

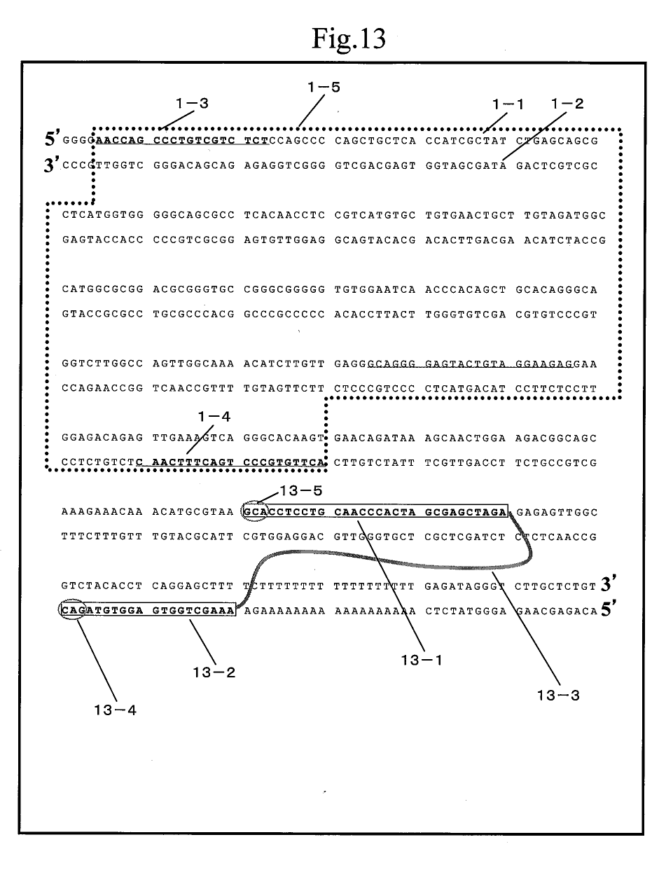

The above effects can be applied for amplification of a specific gene region carried out in a PCR reaction and the like. In this Example, the case of the use of a gene sequence (TP53 gene exon 5: SEQ ID NO: 1) shown in FIG. 13 is described. An amplification region represented by 1-5 was subjected to PCR by allowing primers (second probes) designed as designated by 1-3 and 1-4 to act on template double-strand DNA comprising 1-1 and 1-2. The PCR was carried out under the same conditions used in [Example 1]. In such case, when the amount of template DNA was 1×10⁻²² mol and the amount of primer was 3.4 pmol per reaction, the results were compared with each other, such results being obtained with or without the simultaneous addition of a first probe comprising a 1^{st} sequence 13-1, a 2^{nd} sequence 13-2, and a 3^{rd} sequence 13-3 (GATCTGCGATCTAAGTAAGCTTGGC (SEQ ID NO: 2)) in an amount of 6.8 pmol.

The first probe was modified so as not to act as a primer by reacting either one of second probes (a forward primer 1-3 and a reverse primer 1-4) in a manner such that the 5′ end 13-5 (of the 1^{st} sequence 13-1) originally comprising a sequence 5′-GCA-3′ was substituted with a mismatch sequence 5′-TCT-3′. Further, the 3′ end 13-4 (of the 2^{nd} sequence 13-2) originally comprising a sequence 3′-CAG-5′ was substituted with a mismatch sequence 3′-GAC-5′. Similar effects can be obtained by binding a sequence that is not complementary to a binding region of a template nucleic acid to the 3′ end of a first probe or modifying or substituting a hydroxyl group of the 3′ end with another functional group.

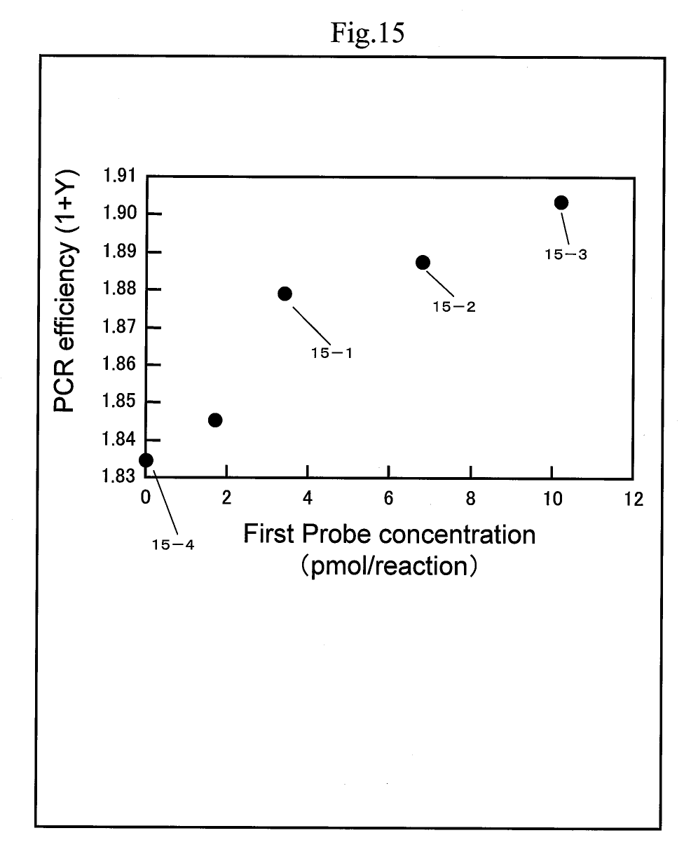

In such case, as designated by 14-1, when a first probe was added during a PCR, a PCR product was obtained in an amount larger than that obtained with the use of a control 14-2 (without the addition of a first probe). A first probe was located downstream of a second probe. Thus, it was almost impossible to expect to obtain effects of the first probe after the 2^{nd} cycle of PCR. Therefore, it was considered that the first probe acted to promote effects of the second probe upon hybridization of the second probe before the 1^{st} cycle. FIG. 15 shows changes in PCR efficiency upon PCR that was carried out with changes to the amount of the first probe from 1.7 pmol, to 3.4 pmol, 6.8 pmol, or 10.5 pmol when the amount of template DNA was 1×10⁻²² mol. The relationship among the PCR cycle number (n), template amount (N₀), PCR product amount (N_{f}), and PCR efficiency (1+Y) is expressed with “N_{f}═N₀×(1+Y)ⁿ” in an exponential amplification region. When the first probe concentration was 3.4, 6.8, or 10.2 pmol (corresponding to 15-1, 15-2, or 15-3), the PCR efficiency was changed significantly compared with that derived from a control 15-4 (without the addition of a first probe).

A real-time PCR method is a method of quantitative evaluation of a PCR product wherein a thermal cycler and a spectrophotometer are integrated such that electrophoresis is omitted. As a method for quantifying a PCR product, a method using specific intercalation of SYBR Green into a groove in a double-strand DNA spiral is conveniently and widely used. Due to PCR characteristics, the PCR product amount can be increased 2 times at maximum with the addition of a single cycle. That is, upon real-time observation of PCR, if conditions such as the initial template amount and the primer concentration are the same, larger final amount of a PCR product, the smaller cycle number at the appearance of the upward curve. Thus, it was considered that an increase in the PCR product amount resulting from the effects of the present invention (corresponding to an increase in PCR efficiency) could be observed also upon real-time PCR.

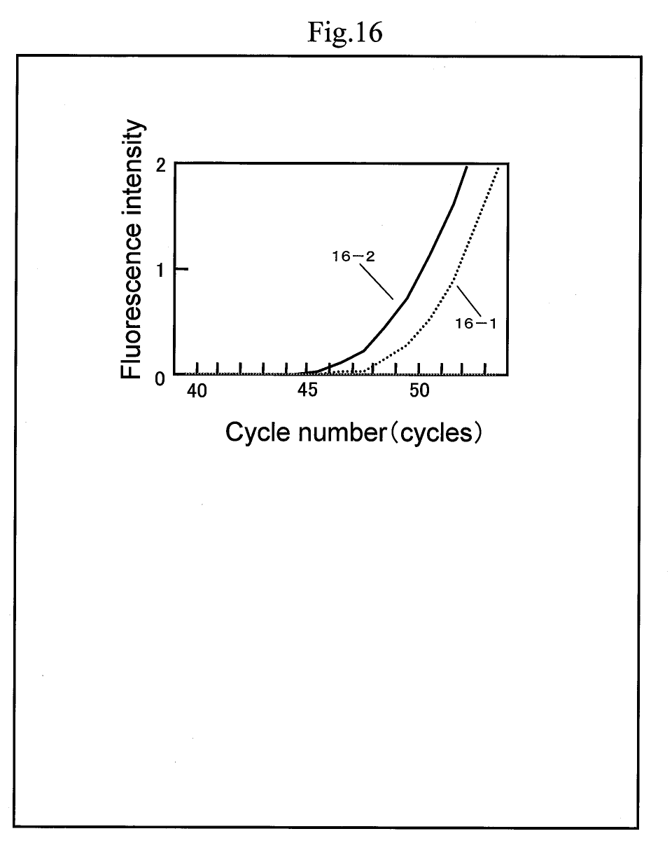

FIG. 16 shows the results of real-time PCR with the addition of template DNA in an amount of 1×10⁻²² mol and a first probe in an amount of 6.8 pmol per reaction. In the case of a control 16-1 (without the addition of a first probe), the upward curve appeared at the average cycle number of 47.45. Meanwhile, in the case involving the addition of a first probe (16-2), the upward curve appeared at the average cycle number of 45.74. Based on the relationship between the initial template amount of a standard sample and the cycle number at which the upward curve appeared, the template concentration in the case of 16-2 was obtained by back calculation. The result was approximately 2×10⁻²² mol, which was 2 times as large as the real template amount of 1×10⁻²² mol. In view of the above, it was considered that a first probe influenced the hybridization efficiency of primers during the 1^{st} cycle of PCR as described above. Specifically, it was considered that a first probe at a concentration 2 to 5 times greater than that of a primer (second probe) was hybridized with template double-strand DNA subjected to thermal denaturation before the primer was hybridized to the same such that the higher-order structure of the DNA was destroyed, resulting in ease of primer hybridization.

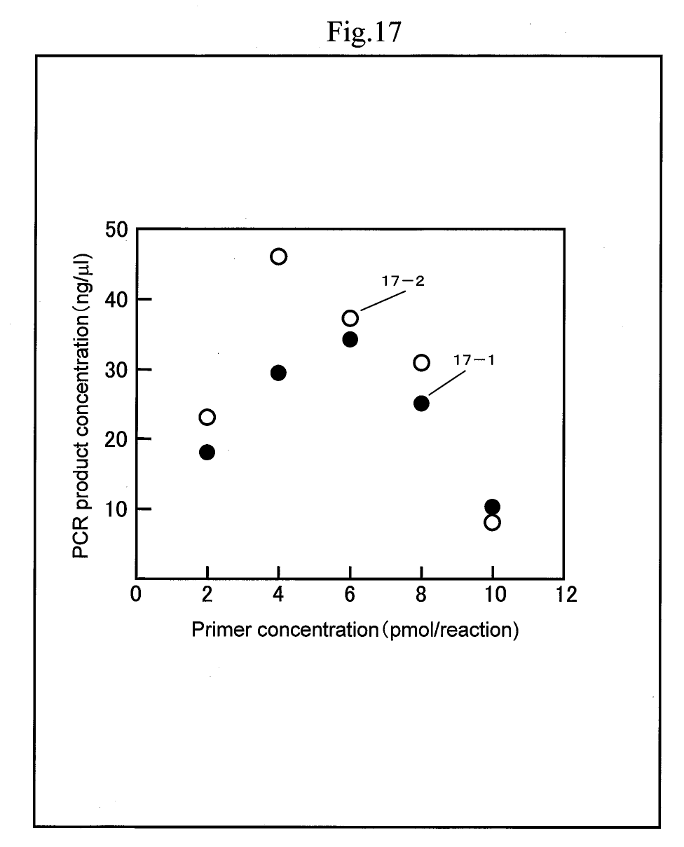

When a first probe was added during a PCR reaction, effects as shown in FIG. 17 were obtained. That is, compared with the results obtained in a general PCR reaction without the addition of a first probe (17-1), a PCR product in a larger amount was obtained at a lower primer concentration in the case involving the addition of a first probe as designated by 17-2. The first probe used in the above experiment is designed so as to be outside of a PCR primer set (second probes). The first probe merely exhibits promotion effects in the 1^{st} primer hybridization reaction. However, when a first probe is designed so as to be inside of a primer set, it is expected that the first probe functions to promote a primer hybridization reaction in the subsequent cycle reactions. In addition, a first probe may function so as to be hybridized with at least two regions of template nucleic acid. Also, when a first probe may function so as to be hybridized with at least two regions of template nucleic acid, a first probe having a structure designated by 3-2, 5-9, 5-11, or 6-1 can be used.

In this Example, PCR using a thermal cycler is described. However, also in the case of complementary strand synthesis in which a single primer is complementary to either strand of template double-strand nucleic acid, it is possible to carry out a complementary strand synthesis reaction by similar operations. Thus, an elongation product can be amplified. Also, such operations are advantageous in that they can be widely used in an amplification method such as an NASBA method or rolling cycle method, wherein a reaction comprising hybridizing a primer with a priming site is carried out and a complementary strand synthesis reaction is carried out with the use of an enzyme reaction caused by polymerase and the like. Alternatively, such operations can be applied to an isothermal elongation reaction (at a constant temperature of 37° C., for example) with the use of an enzyme such as Escherichia coli DNA polymerase I or a Klenow fragment, which is a partial enzyme of such polymerase, and an isothermal amplification method such as an ICAN method (TaKaRa) or a LAMP method (Eiken Chemical Co., Ltd.). In such case, a template is first hybridized with a first probe.

### Example 5

### Application of the Present Invention to Mutation Analysis (BAMPER Method)

The BAMPER (Bioluminometric Assay coupled with Modified Probe Extension Reactions) method (Guo-hua Zhou, et al., Nucleic Acid Research, 29, e93 (2001)) involves a technique suitable for detection of mutations found in individual specimens. The method has been examined for practical application (Y Nakashima, et al., Clinical Chemistry, 50, 8, 1417-1420 (2004)). The method is based on mutation analysis technology using bioluminescence. According to the method, an elongation reaction is carried out with the use of 2 to 4 types of probes (having A, G, C, or T at the 3′ ends thereof) corresponding to mutation sites such that the elongation reaction proceeds only when a probe corresponding to the base type of a detection site is used. Pyrophosphoric acid generated as a result of the reaction is transformed into ATP. Then, luminescence is caused by a luciferin-luciferase reaction. The base type can be detected by measuring such luminescence within several minutes. This simple method is useful for detection of point mutations, insertion or deletion of at least one base, or single nucleotide polymorphism (SNP) generally observed in cancer tissue cells. The procedures are as follows.

First, a gene sequence to be analyzed is amplified by a PCR or the like. Next, a PCR product of interest is purified in order to remove primers and dNTPs used for a PCR by enzymatic cleanup. Specifically, a solution obtained after a PCR reaction (15 μL) is mixed with shrimp alkaline phosphatase at a concentration of 1 unit/μL, (0.7 μL), exonuclease I at 10 unit/μL (0.06 μL), a 10×PCR buffer (Amersham Pharmacia) (0.3 μL), and sterilized water (3.94 μL). After an enzyme reaction involving incubation at 37° for 40 minutes, enzymes are inactivated by heating at 80° C. for 15 minutes. The obtained reaction solution is dispensed into each well of a 96-well PCR plate (in white color) (3 each). A mutation-specific probe (1 μL: 5 pmol/μL) is added thereto. Taq. DNA polymerase (0.0275 μL: 5 unit/μL), and 5 mM dNTPs (from which pyrophosphoric acid has been removed in advance) (0.04 μL) are first mixed. Then, sterilized water is added to a mixture such that 1.0 μL of the resultant is obtained. The thus obtained solution (1.0 μL) is added to each well. Mineral oil (4 μL) is applied thereto such that multilayers are formed. A cycle of 94° C. for 10 seconds and 60° C. for 10 seconds is repeated 5 times, followed by cooling to 25° C. A luminescence reagent at room temperature (a bioluminescence kit for detection of ATP with the use of a firefly-derived luciferase (such ATP being obtained by transforming pyrophosphoric acid into ATP): see T Sakakibara, et al., Analytical Biochemistry, 268, 94-101 (1999)) (10 μL) is added to each well, followed by mixing by pipetting. Then measurement is carried out with a luminometer. Accordingly, it is possible to detect with ease whether or not an elongation reaction has taken place based on luminescence intensity depending on the amount of pyrophosphoric acid.

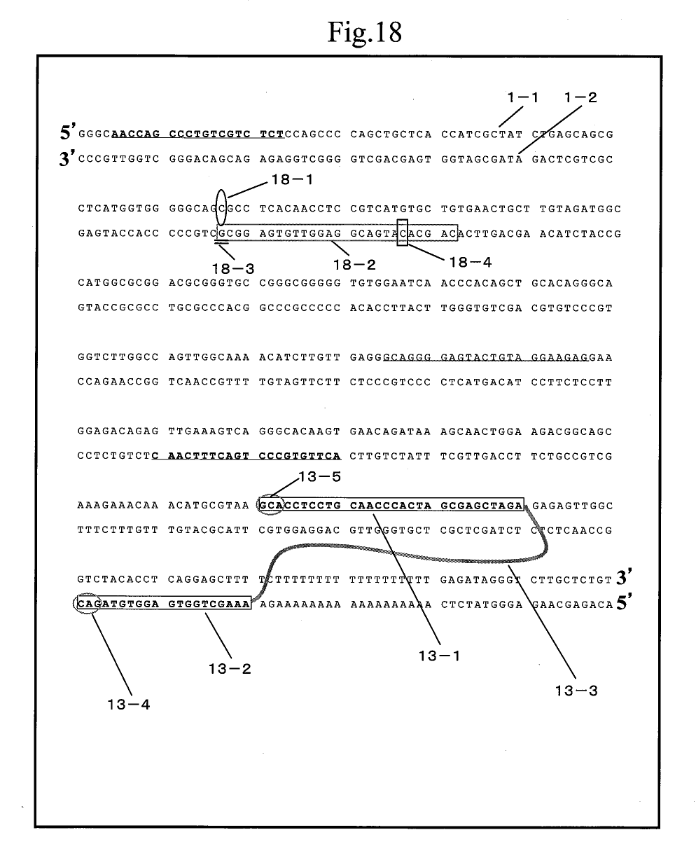

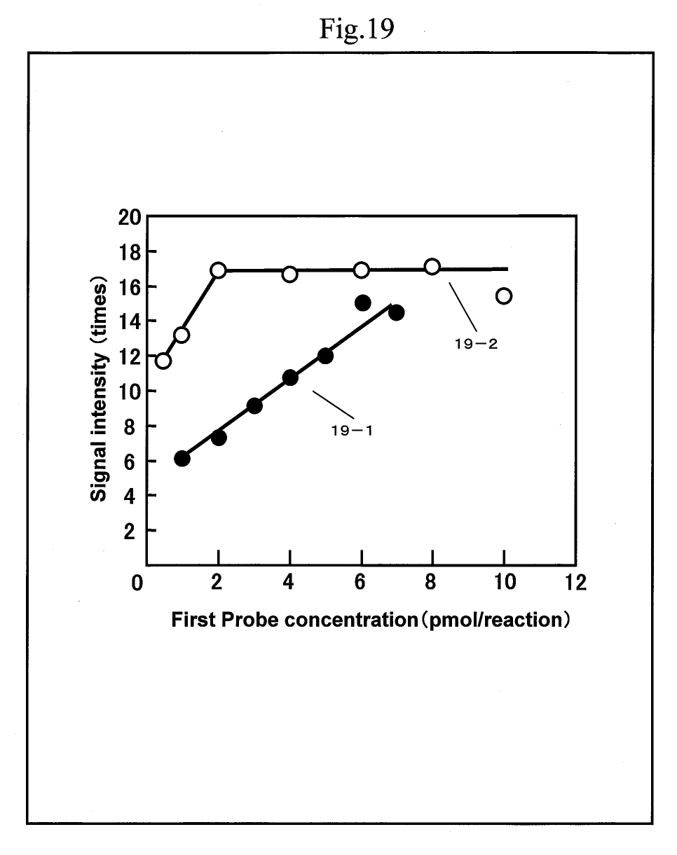

An example of application of the present invention to the above BAMPER method is described below. A double-strand template DNA (TP53 gene exon 5) comprising 1-1 and 1-2 shown in FIG. 18 was amplified by PCR. It has been known that a base 18-1 in a nucleic acid strand 1-1, which is the second base (C: standard type) of 3 bases constituting the 175^{th} codon of the gene, is substituted with T (mutation type) in many cancers. A second probe sequence used for detection of such mutation is represented by 18-2, in which the 3′ end (18-3) is G or A, which corresponds to C or T described above. In order to avoid formation of a holding structure of the second probe as a result of intramolecular hybridization, a mismatch base was inserted into 18-4 and the original base C was substituted with G. FIG. 19 shows relationship between elongation (shown as signal intensity) and concentration dependence of the second probe with or without the simultaneous addition of a first probe comprising a 1^{st} sequence 13-1, a 2^{nd} sequence 13-2, and a 3^{rd} sequence 13-3 when a mutation 18-1 was detected with the use of the thus obtained second probe 18-2. In such case, the first probe was modified so as not to act as a primer by reacting a second probe 18-2 in a manner such that the 5′ end 13-5 (of the 1^{st} sequence 13-1) originally comprising a sequence 5′-GCA-3′ was substituted with a mismatch sequence 5′-TCT-3′. Further, the 3′ end 13-4 (of the 2^{nd} sequence 13-2) originally comprising a sequence 3′-CAG-5′ was substituted with a mismatch sequence 3′-GAC-5′.

As shown with 19-1, when a second probe alone was allowed to react at 60° C., the detection signal also increased within the range of 1 to 6 pmol in a concentration-dependent manner. On the other hand, when the first probe was allowed to coexist (19-2), a strong signal intensity, which was 17 times greater than that of the strongest signal (15 times greater than the signal of a control at 6 pmol) obtained with the use of the second probe alone, was observed within the lower concentration range. In addition, it was possible to maintain such signal intensity within the range of 2 to 8 pmol. That is, it was considered that reassociation of a template double-strand subjected to thermal denaturation was inhibited as a result of the coexistence of the second probe and the first probe, resulting in the improvement of the hybridization efficiency of the second probe. Thus, it became possible to detect a mutation 18-1 at a lower concentration of a second probe than in the case of measurement with the presence of a second probe alone. A structure designated by 3-2, 5-9, 5-11, or 6-1 described above is applied to a first probe.

In addition to the above method of detecting pyrophosphoric acid, wherein pyrophosphoric acid is transformed into ATP, ATP is used as an energy source for a luciferin-luciferase reaction and luminescence is detected, the following method or the like can be applied: a method wherein pyrophosphoric acid is transformed into formazan, hydrogen peroxide, superoxide, carbon dioxide, L-phenylalanine, sulphate ion, or the like by a variety of general chemical reactions and the resultant is detected using a detector or by visual observation (see JP Patent Publication (Kokai) No. 2003-174900 A). Alternatively, a method wherein a substance that emits fluorescence when losing metal ions functioning as quenchers is detected using a detector or by visual observation, such loss of the metal ions resulting from a binding action between the metal ions and metal ions of pyrophosphoric acid that has been generated as a result of an elongation reaction caused by mutation-specific primers, can also be applied.

Examples of other general mutation analysis methods include a one base elongation method, an Invader (registered trademark) method (Third Wave Technologies), a MassArray™ method (Sequenom), an ASP-PCR method (TOYOBO), a UCAN method (TaKaRa), an STA method (Mutector (registered trademark) kit, TRIMGEN), and a PCR-PHFA method (Roche Diagnostics; K-ras codon 12 mutation detection kit). Each method comprises a step of hybridizing a probe designed to have a 3′ end that is complementary to a mutation base with sample DNA. In general, when gene analysis comprises a step of hybridizing a probe with a sample in a specific manner, it is necessary to prepare excessive amounts of probes relative to the amount of a sample. When a hybridization reaction is caused on a solid phase, it is difficult to detect signals in some cases because reaction efficiency becomes significantly poor compared with cases involving reactions using a liquid phase. However, the present invention can also be applied to such case. In the cases of isothermal reactions involving a UCAN method or an STA method, an interference oligo may previously be hybridized with a sample template in the same manner used in the above ICAN method or LAMP method.

All publications, patents, and patent applications cited herein are incorporated herein by reference in their entirety.

### INDUSTRIAL APPLICABILITY

In the field of medical practice, analyses of gene mutation such as point mutation (representative example) occurring in a lesion over the course of onset and development of a disease have been actively conducted. Along with accumulation of genetic information, for instance, the relationship between changes in specific bases found in a specific site on a specific gene and progression of a disease is being revealed. The present invention is applied to the technology for DNA chips that are widely used for such mutation analysis and gene expression analysis for specific diseases when it is necessary to allow a small amount of a nucleic acid sample to react with a detection probe with good efficiency. Alternatively, in order to carry out the above test with good sensitivity and good efficiency, it is essential to carry out amplification of a specific gene fragment by a PCR method or the like. In such a case, it is highly significant to stably amplify a sample gene fragment with the use of a small amount of DNA as conducted in the present invention. Thus, it is believed that the present invention is very useful in the medical industry.

### [Free Text of Sequence Listing]

SEQ ID NO: 1—a part of the TP53 gene exon 5 shown in FIGS. 1, 13, and 18
SEQ ID NO: 2—3^{rd} sequences (1-12) and (13-3)

1. A method of analyzing nucleic acid comprising: a step of hybridizing a first probe comprising a 1^{st} sequence complementary to one strand of double-strand nucleic acid, a 2^{nd} sequence complementary to the other strand thereof, and a 3^{rd} sequence that binds the 1^{st} sequence and the 2^{nd} sequence; and a step of hybridizing at least one type of a second probe with the double-strand nucleic acid.

2. The method of analyzing nucleic acid according to claim 1, wherein the 3^{rd} sequence constituting the first probe is 10 mer to 100 mer and is not complementary to either sequence of the double-strand nucleic acid.

3. The method of analyzing nucleic acid according to claim 1, wherein the binding region of the second probe on the double-strand nucleic acid is located between the binding region of the 1^{st} sequence and the binding region of the 2^{nd} sequence on the double-strand nucleic acid.

4. The method of analyzing nucleic acid according to claim 3, wherein the 1^{st} sequence and the 2^{nd} sequence are each hybridized with a region that is at least 10 bases away from the end of the binding region of the second probe on the double-strand nucleic acid.

5. The method of analyzing nucleic acid according to claim 1, wherein the 3^{rd} sequence that constitutes the first probe forms a three-dimensional structure in a loop form as a result of intrastrand hybridization.

6. The method of analyzing nucleic acid according to claim 1, wherein:
two types of first probes are used; and
the two types of first probes that have been separately hybridized with neighboring regions on the double-strand nucleic acid form a complementary strand bond between their 3^{rd} sequences.

7. The method of analyzing nucleic acid according to claim 1, wherein the double-strand nucleic acid is quantified by measuring the amount of the second probe hybridized with the double-strand nucleic acid.

8. The method of analyzing nucleic acid according to claim 7, wherein the amount of the second probe hybridized with the double-strand nucleic acid is quantified based on the amount of fluorescence or radiation obtained by labeling the second probe with a phosphor or a radioactive isotope or based on the amount of luminescence or color development resulting from a reaction between an enzyme and a substrate thereof obtained by labeling the second probe with the enzyme selected from the group consisting of alkaline phosphatase, peroxidase, β-galactosidase, and luciferase

9. The method of analyzing nucleic acid according to claim 1, further comprising a step of carrying out a complementary strand elongation reaction using the second probe hybridized with the double-strand nucleic acid.

10. The method of analyzing nucleic acid according to claim 9, wherein the first probe has a structure in which complementary strand elongation does not take place at the 3′ end thereof.

11. The method of analyzing nucleic acid according to claim 9, wherein the first probe has a structure in which at least one base of three bases at the 3′ end thereof is mismatched with a binding region of the first probe on the double-strand nucleic acid.

12. The method of analyzing nucleic acid according to claim 9, wherein a sequence that is not complementary to the binding region of the first probe on the double-strand nucleic acid is added to the 3′ end of the first probe.

13. The method of analyzing nucleic acid according to claim 9, wherein a hydroxyl group of at least one base of three bases at the 3′ end of the first probe is modified or substituted with another functional group.

14. The method of analyzing nucleic acid according to claim 9, wherein the second probe is immobilized on a solid phase.

15. The method of analyzing nucleic acid according to claim 9, comprising: a step of simultaneously adding the first probe and the second probe to a nucleic acid sample containing double-strand nucleic acid that is expected to have a mutation, such probes each being hybridized at the 3′ end thereof with a candidate region for the mutation; a step of carrying out an elongation reaction with the use of the hybridized first and second probes; and a step of judging whether or not the double-strand nucleic acid sample has a mutation site based on the results of the elongation reaction.

16. The method of analyzing nucleic acid according to claim 15, wherein an elongation reaction that is carried out with the use of a second probe is a single base elongation reaction and the type of base to be introduced is identified upon the reaction, thus making it possible to judge whether or not a neighboring base of the 3′ end of the second probe has a mutation.

17. The method of analyzing nucleic acid according to claim 15, wherein the elongation reaction is induced when at least two bases of the double-strand nucleic acid are complementary to at least two bases that exist at the 3′ end of the first probe.

18. The method of analyzing nucleic acid according to claim 15, wherein the second probe has a structure that is mismatched with a binding region on the double-strand nucleic acid.

19. The method of analyzing nucleic acid according to claim 1, comprising a step of amplifying at least a partial region of the double-strand nucleic acid with the use of the second probe comprising a pair of an upstream primer and a downstream primer used for amplification, wherein
the 1^{st} sequence and the 2^{nd} sequence are separately hybridized with sequences neighboring or containing the region amplified with the primers on the double-strand nucleic acid.

20. The method of analyzing nucleic acid according to claim 19, wherein the 1^{st} sequence and the 2^{nd} sequence are each hybridized with a region within 500 bases away from the end of the region of the double-strand nucleic acid.


SEQUENCE LISTING (4 sequences)

SEQ ID NO: 1 - DNA, 420 bp, Artificial Sequence
  Description of Artificial Sequence Synthetic polynucleotide
gggcaaccagccctgtcgtctctccagccccagctgctcaccatcgctatctgagcagcgctcatggtgggggcagcgcctcacaacctccgtcatgtgctgtgactgcttgtagatggccatggcgcggacgcgggtgccgggcgggggtgtggaatcaacccacagctgcacagggcaggtcttggccagttggcaaaacatcttgttgagggcaggggagtactgtaggaagaggaaggagacagagttgaaagtcagggcacaagtgaacagataaagcaactggaagacggcagcaaagaaacaaacatgcgtaagcacctcctgcaacccactagcgagctagagagagttggcgtctacacctcaggagcttttcttttttttttttttttttgagatagggtcttgctctgt

SEQ ID NO: 2 - DNA, 25 bp, Artificial Sequence
  Description of Artificial Sequence Synthetic oligonucleotide
gatctgcgatctaagtaagcttggc

SEQ ID NO: 3 - DNA, 15 bp, Artificial Sequence
  Description of Artificial Sequence Synthetic oligonucleotide
ttgactacccatgaa

SEQ ID NO: 4 - DNA, 16 bp, Artificial Sequence
  Description of Artificial Sequence Synthetic oligonucleotide
ttcatgggtagtcaag